(19) United States
(12) Patent Application Publication
(10) Pub. No.: US 2022/0170145 A1
(43) Pub. Date: Jun. 2, 2022

(54) TREATMENT METHOD FOR ZIRCONIUM ALLOY AND APPLICATION

(71) Applicant: SUZHOU MICROPORT ORTHORECON CO., LTD., Suzhou, Jiangsu (CN)

(72) Inventors: Yu SHEN, Suzhou, Jiangsu (CN); Tianbai YU, Suzhou, Jiangsu (CN); Chih-Hsin WENG, Suzhou, Jiangsu (CN)

(21) Appl. No.: 17/436,974

(22) PCT Filed: Nov. 13, 2019
(86) PCT No.: PCT/CN2019/117752

§ 371 Date: Sep. 7, 2021

(30) Foreign Application Priority Data
Mar. 7, 2019 (CN) 201910172951.X

(51) Int. Cl. C23C 8/10; C23C 8/42; C23C 8/80; A61L 27/04; A61L 27/30

(52) U.S. Cl. CPC C23C 8/10; C23C 8/42; A61L 2430/24; A61L 27/047; A61L 27/306; C23C 8/80

(57) ABSTRACT

A treatment method for zirconium alloy includes performing a surface layer oxidation and removal treatment on a surface layer of zirconium alloy. The surface layer oxidation and removal treatment comprises performing an oxidation treatment on the surface layer of the zirconium alloy to obtain an oxide surface layer, and then removing the oxide surface layer to expose a metal substrate. A method for fabricating a surface oxide ceramic layer of zirconium alloy and a material for a medical implant are also provided.

### TECHNICAL FIELD

The present application relates to the field of materials for medical implants, in particular, to a treatment method for a zirconium alloy and application thereof.

### BACKGROUND

Materials for use in medical implants are required to have high strength, corrosion resistance and histocompatibility. This makes only a part of metal alloys to be desirable materials meeting above requirements, such as 316L stainless steel, cobalt-chromium-molybdenum alloys, titanium alloys, as well as zirconium alloys that have been recognized in recent years as the most suitable materials for manufacturing bearing and non-bearing prostheses.

In general, a zirconium alloy has a relatively soft surface with a hardness that range from 1.5 GPa to 3 GPa, making it vulnerable to abrasion by harder third-body particles and thus owing a poor wear-resisting property. Traditionally, the surface hardness of the zirconium alloy is usually improved by surface oxidization or surface nitriding in prior art. The principle of surface oxidization is to form an oxide ceramic surface on the zirconium alloy. The surface hardness for zirconium oxide may be up to 12 GPa. FIG. 1 shows a structural model of an oxide ceramic surface. As shown, the oxide ceramic surface layer usually has a thickness of 5-6 μm and the oxygen-rich diffusion layer having a thickness of about 1.5-2 μm is present at the interface between the oxide ceramic surface layer and the metal substrate. Such structure enables to offer a hard ceramic surface as well as keep the good plasticity of the zirconium alloy substrate, thereby resulting in improved surface resistance to wear and scratching and avoiding the risk of brittle cracking arising from the use of ceramic materials in manufacturing prostheses.

U.S. Pat. Nos. 2,987,352 and 3,615,885 each describe the approach of heating the zirconium alloy in air to form an oxide ceramic surface layer thereon. The surface of a medical implant treated by such approach owns excellent resistances to wear, scratching and brittle cracking, thereby showing the good effect of such approach. At present, the zirconium alloy material used in practical manufacturing is originated from the zirconium alloy material used in nuclear industry, which is surface oxidized to form an oxide ceramic surface layer with a color of dark blue. The oxide ceramic surface layer with a color of dark blue has a high compactness and few cracks. However, the final products are very expensive due to the very high price of raw material.

If the zirconium alloy material used in general industries and having a much low price is adopted to produce the medical implant having an oxide ceramic surface layer, they would produce the oxide ceramic surface layer with a color of grey white even treated with a same processing. It has been found in practice that the oxide ceramic layer with a color of grey white is bad in compactness and bonding strength and some of them are even found with shedding of oxide particles from the surface during the preparation of cross-sectional samples. Since the exfoliated oxide particles from the surface have very high hardness, such type of oxide ceramic surface layer cannot improve wear resistance of the zirconium alloys, but accelerate the rate of wear due to the generation of a huge amount of exfoliated oxide particles, which results in its inapplicability for the manufacture of medical implants.

Therefore, it would be desirable to develop a method capable of replacing the expensive zirconium alloy material used in the nuclear industry with the zirconium alloy material used in general industries to lower the cost under the premise that the performance of the produced oxide ceramic layer meets the requirements.

### SUMMARY

The inventors have found through painstaking researches that a low compactness of an oxide ceramic layer for the zirconium alloy is attributable to an excessive content of hafnium element in the zirconium alloy material, which causes a large amount of micro-cracks to be produced in the oxide ceramic layer. Present application provides a method for reducing the content of hafnium element on the surface of the zirconium alloy and the application thereof to solve the problems of the large amount of micro-cracks and the low compactness of oxide ceramic layer for the zirconium alloy.

To solve the above technical problems, technical solution of present application provides:

a method of treating a zirconium alloy, comprising: a step of performing a surface layer oxidation and removal treatment on the zirconium alloy, where the surface layer oxidation and removal treatment comprises: performing an oxidation treatment on a surface layer of the zirconium alloy to obtain an oxide surface layer; and performing a removal treatment to the oxide surface layer to expose a metal substrate.

Optionally, the zirconium alloy has an initial content of hafnium element ranging from 0.5 wt % to 8 wt %.

Optionally, the oxidation treatment is conducted at a temperature of 500° C. to 700° C. and a treatment time of 0.5 h to 10 h.

Optionally, the oxide surface layer is removed by grinding, fine machining, mechanical polishing, vibratory polishing or any combination thereof.

Optionally, a thickness of the oxide surface layer removed in the removal treatment ranges from 1 μm to 20 μm.

Optionally, the thickness of the oxide surface layer removed in the removal treatment ranges from 3 μm to 12 μm.

Optionally, the method further comprises repeating the step of performing a surface layer oxidation and removal treatment for 1 to 5 times.

The present application also provides a method for producing an oxide ceramic layer on a surface of a zirconium alloy, comprising treating the zirconium alloy with the above described method; and performing an oxidation treatment on a surface of the exposed metal substrate.

Optionally, the oxide ceramic layer has a content of hafnium element ranging from 0.3 wt % to 6 wt %.

The present application also provides a material for use in medical implants comprising a metal substrate, an oxygen-rich diffusion layer and an oxide ceramic layer, the metal substrate made of a zirconium alloy, where a content of hafnium element in the metal substrate is higher than a content of hafnium element in the oxide ceramic layer.

Optionally, the content of hafnium element in the metal substrate ranges from 0.5 wt % to 8 wt %, and the content of hafnium element in the oxide ceramic layer ranges from 0.3 wt % to 6 wt %.

The technical solution of present application is able to lower the contents of hafnium oxides in the oxide ceramic surface layer of the zirconium alloy having a high content of hafnium element, so as to solve the problem of micro-cracks in the oxide ceramic surface layer of the high-hafnium-content zirconium alloy, and thus offer the oxide ceramic layer improved abrasive resistance, hardness and damage resistance. Moreover, this method is simple and has a low cost. In addition, compared with the method using expensive low-hafnium-content zirconium alloy as the raw material, the method provided in present application is able to achieve the oxide ceramic layer with comparable performance as well as dramatically reduce the cost of raw material, thereby making it more competitive in the marketplace. In particular, the method provided in present application is suitable for the surface treatment of a material for use in joint prostheses.

### BRIEF DESCRIPTION OF THE DRAWINGS

FIG. 1 schematically illustrates a cross-sectional structure of an oxide ceramic surface layer formed on a zirconium alloy in prior art.

FIG. 2 is a flowchart graphically illustrating a process for reducing the content of hafnium oxide in an oxide ceramic surface layer on a zirconium alloy according to a particular embodiment.

FIG. 3 is a Scanning Electron Microscope (SEM) image showing the cross section of an oxide ceramic layer of Sample 1 according to Embodiment 1.

FIG. 4 is a Scanning Electron Microscope (SEM) image showing the cross section of an oxide ceramic layer of Sample 2 according to Embodiment 1.

FIG. 5 is a Scanning Electron Microscope (SEM) image showing the cross section of an oxide ceramic layer of Sample 3 according to Embodiment 1.

FIG. 6 shows hardness curve graphs of the oxide ceramic layers of Samples 1 to 3 according to Embodiment 1.

FIG. 7 shows an indentation photo of Sample 2 according to Embodiment 1 created by a Rockwell diamond indenter under a load of 60 kg.

FIG. 8 shows an indentation photo of Sample 3 according to Embodiment 1 created by a Rockwell diamond indenter under a load of 60 kg.

FIG. 9 is a metallurgical microscope image showing the cross-sectional morphology of an oxide ceramic layer of Sample 4 according to Embodiment 2.

FIG. 10 is a metallurgical microscope image showing the cross-sectional morphology of an oxide ceramic layer of Sample 5 according to Embodiment 2.

FIG. 11 is a metallurgical microscope image showing the cross-sectional morphology of an oxide ceramic layer of Sample 6 according to Embodiment 2.

FIG. 12 shows hardness curve graphs of the oxide ceramic layers of Samples 4 to 6 according to Embodiment 2.

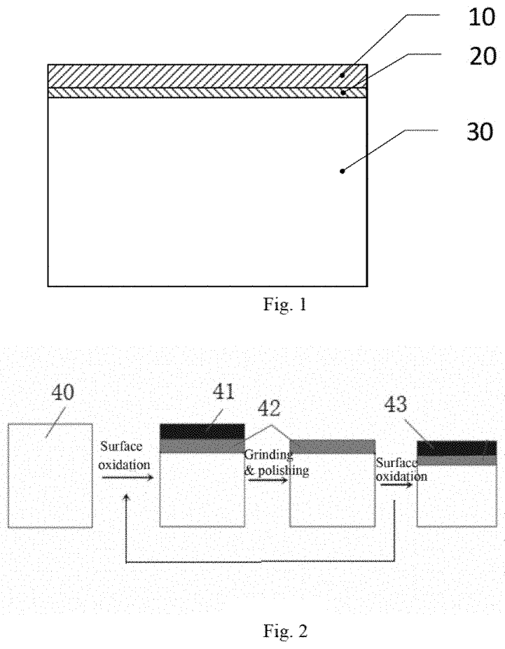

In the figures:

10, oxide ceramic surface layer; 20, oxygen-rich diffusion layer; 30, metal substrate; 40, first surface layer; 41, first oxide surface layer; 42, second surface layer; and 43, second oxide surface layer.

### DETAILED DESCRIPTION

The inventors have found through investigations and researches that the zirconium alloy material inevitably contains a certain quantity of hafnium element as an impurity because the two elements coexist in nature. In zirconium ores, the ratio of the weight percentage of hafnium element to the weight percentage of zirconium element generally ranges from 1.5% to 3.0%. Due to the very close properties of hafnium and zirconium elements, it is difficult to separate hafnium element from the zirconium element. Existing techniques for separating hafnium from zirconium are all costly and tend to cause environmental pollution. In common industrial applications, as the presence of hafnium does not affect the mechanical and chemical properties of alloys, removal of hafnium element generally is unnecessary. However, in the nuclear industry, as the hafnium element has a large thermal neutron absorption area, the presence of hafnium impedes the use of the zirconium alloys as a cladding material in nuclear industry. Therefore, it is necessary to separate hafnium from the zirconium alloy to produce the zirconium alloy with a very low content (<0.005%) of hafnium element.

Through detections of oxide ceramic surface layers, it has been found that the surface and interior of the oxide ceramic layer produced from zirconium alloy material for use in general industries have numerous micro-cracks. The contents of various major elements in the oxide ceramic layer have been measured on the surface and at different cross-sectional depths of the oxide ceramic layer. It has been found from the measurement results that hafnium oxide is widely distributed within the oxide ceramic layer.

From the above facts, the inventors speculate that the primary reason for the decreased compactness of the oxide ceramic layer is the formation of micro-cracks in the oxide ceramic layer caused by the hafnium oxide in the oxide ceramic layer.

The contents of various major elements have been measured on the surfaces of the metal substrate of the oxidized zirconium alloy, oxygen-rich diffusion layer and oxide ceramic surface layer and at different cross-sectional depths of the oxide ceramic surface layer. It has been found from the measurement results that although hafnium oxide is widely distributed within the oxide ceramic layer, the content of hafnium element in the zirconium alloy substrates near the oxide ceramic layer goes down.

The principle for this phenomenon may be as follow. In oxidation, the Gibbs free energy for the formation of hafnium dioxide is −1087.2 kJ/mol, and the Gibbs free energy for the formation of zirconium dioxide is −1038.7 kJ/mol. Since the Gibbs free energy for the formation of hafnium dioxide is lower than that for the formation of zirconium dioxide, the hafnium element is easier to combine with oxygen than the zirconium element. The hafnium atom preferentially binds to oxygen in oxidation and thus enriches on the surface. This results in a decreased content of hafnium element in the zirconium alloy substrates near the oxide ceramic layer.

Accordingly, after the first surface oxidation, the oxide surface layer (i.e., the oxide ceramic layer and oxygen-rich diffusion layer) with enriched hafnium element is ground and polished to the near-surface substrate having a decreased content of hafnium element. Then, the second surface oxidation is performed on such surface (i.e., the near-surface substrate) to obtain the oxide ceramic surface layer with reduced content of hafnium element, thereby reducing micro-cracks within oxide ceramic surface layer and improving quality of the oxide ceramic surface layer. Although such method can only reduce the content of hafnium on the surface, it is totally suitable as the material for use in medical implant as the presence of hafnium inside the alloy does not affect the performance of the medical implants. Besides, compared with the method using zirconium alloy for the nuclear industry as raw material, this method is advantageous in easier availability of raw material and much lower cost.

Specifically, present application prepares a material for use in medical implants by the following method. Such material includes a metal substrate 30, an oxygen-rich diffusion layer 20 and an oxide ceramic layer 10 that are arranged from inside to outside. The metal substrate 30 is a zirconium alloy that is not surface oxidized. The oxide ceramic layer 10 is a layer in which oxygen element is present essentially in the form of the oxide. The oxygen-rich diffusion layer 20 is a layer in which the content of oxygen is higher than the content of oxygen in metal substrate and the oxygen element is present essentially in the form of solute atoms. Hafnium is present in the metal substrate 30 at an amount of 0.5 wt % to 8 wt %. The oxide ceramic layer 10 has a lower content of hafnium than the metal substrate 30, which is 0.3 wt %-6 wt %, preferably 0.3 wt %-2 wt %, more preferably 0.3 wt %-1 wt %, e.g., 0.4 wt %, 0.5 wt %, 0.7 wt % or 0.9 wt %.

As shown in FIG. 2, steps of the method for preparing a material for use in medical implants are as follows:

(1) Performing a surface oxidation treatment on the first surface layer 40 of a zirconium alloy metal substrate 30 containing a hafnium content of 0.5 wt % to 8 wt %. The surface oxidation treatment may be performed in air or in any other oxygen-containing atmosphere. Alternatively, it may also be performed using a vapor, a water bath or a salt bath. The surface oxidation process may be performed at a oxidation temperature of 500° C. to 700° C., preferably 550° C. to 600° C., for a treatment time of 0.5-10 h, preferably 4-6 h. The first oxide surface layer 41 enriched with hafnium therein is formed on the surface of the metal substrate 30 after the surface oxidation treatment. The first oxide surface layer 41 consists of an oxygen-rich diffusion layer 20 and an oxide ceramic layer 10.

(2) Removing the first oxide surface layer 41 enriched with hafnium by means of an approach selected from the group consisting of grinding, fine machining, mechanical polishing, vibratory polishing and any combination thereof with a removal thickness of 1-20 μm, preferably 3-12 μm, so as to expose the second surface layer 42 with reduced content of hafnium element. Preferably, the removal thickness is selected as the thickness that is able to exactly remove the first oxide surface layer 41. That is, the removal thickness is selected as the thickness that is able to exactly remove the oxygen-rich diffusion layer 20 and the oxide ceramic layer 10 together with partial metal substrate. The thickness of the oxygen-rich diffusion layer 20 and the oxide ceramic layer 10 each may be determined from a cross-section measurement of the material. This is because both the oxygen-rich diffusion layer 20 and the oxide ceramic layer 10 are distinguishable from a cross section of the material by the naked eye. Alternatively, the thickness may also be speculated based on the used oxidation conditions and previous experience.

(3) Repeating step (1) to perform another surface oxidation treatment to form a second oxide surface layer 43 with a reduced content of hafnium element.

(4) Repeating steps (1) to (3) for one time, or more times, preferably 1-5 times, until the formed oxide ceramic layer has a reduced hafnium content of 0.3 wt % to 6 wt %, preferably 0.3 wt % to 2 wt %, more preferably 0.3 wt % to 1 wt %.

For ease of understanding, the method for reducing the content of hafnium oxide in an oxide ceramic surface layer of a zirconium alloy provided in present application will be described below with reference to several embodiments. It is to be understood that these embodiments are described for the mere purpose of illustration and do not limit the protection scope thereof in any sense.

Unless particularly noted, each material or reagent used in the following embodiments is commercially available, and each process or parameter can be realized by existing technology.

### Embodiment 1

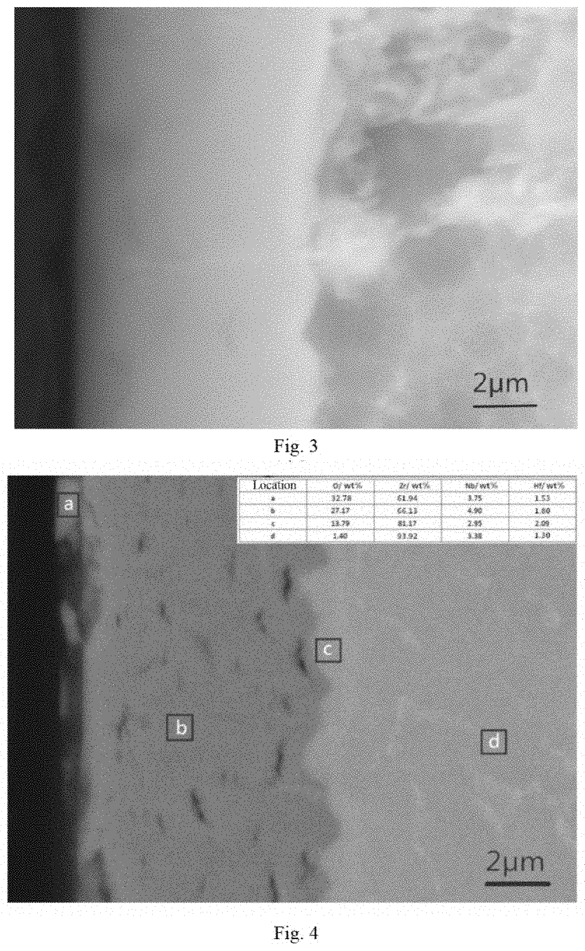

Samples 1 and 2 each with an oxide ceramic surface layer were prepared by heating zirconium alloys with hafnium contents of <0.005% and about 2.26% to 550° C. and maintaining them at the temperature for 6 h in air respectively. Scanning Electron Microscope (SEM) images showing their cross-sectional morphologies were shown in FIGS. 3 and 4. In each of the images, there were an oxide ceramic layer on the left, an oxygen-rich diffusion layer in the middle and a metal substrate on the right. As can be seen in FIG. 3, the oxide ceramic surface layer of Sample 1 has a high compactness with few defects. However, as shown in FIG. 4, there were numerous micro-cracks in the oxide ceramic surface layer of Sample 2. Elemental analysis results of Sample 2 show that oxide ceramic surface layer (1.53 wt % at location a and 1.80 wt % at location b) and the oxygen-rich diffusion layer (2.09 wt % at location c) have high contents of the hafnium element, while the location of the substrate near the oxide ceramic layer (location d) has a hafnium content (1.3 wt %) remarkably lower than the initial hafnium content (2.26 wt %) of the alloy.

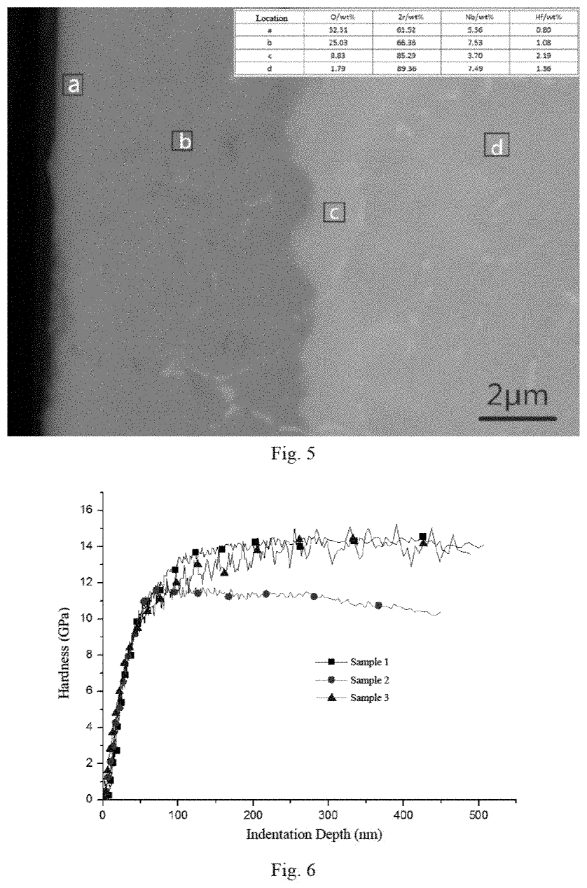

The oxide ceramic surface layer of Sample 2 was removed by means of mechanical grinding, polishing or another approach with a removal thickness of about 10 μm. That is, the oxide ceramic surface layer and the oxygen-rich diffusion layer were removed to expose the surface with a low hafnium content. Subsequently, the surface with a low hafnium content was performed a surface oxidization treatment by heating to 550° C. and maintaining at the temperature for 6 h in air to obtain Sample 3. FIG. 5 shows a cross-sectional morphology and the contents of various elements at different locations of Sample 3. As can be seen in FIG. 5, the oxide ceramic surface layer of Sample 3 obtained by above method exhibits a high compactness without noticeable cracks. Percentages of various major elements at different cross-sectional locations of Samples 2 and 3 measured by a Energy Disperse Spectroscopy (EDS) were listed at upper right corners of FIGS. 4 and 5, respectively. The measurements show a greatly reduced content of hafnium element in the oxide ceramic surface layer (locations a and b) of Sample 3. For the surface layer of Sample 2, the percentage of hafnium in the total content of zirconium, niobium and hafnium is about 2.28 wt %, while for the surface layer of Sample 3 obtained by the above method, the percentage of hafnium in the total content of zirconium, niobium and hafnium is about 1.18 wt %.

FIG. 6 shows hardness curve graphs of the oxide ceramic surface layers of Samples 1 to 3. The hardness curve graph is measured by a nanoindentation instrument in a CSM (Continuous Stiffness Measurement) mode with an indentation depth of up to 500 nm and the plotted values were average hardness measured at indentation depths in the range of 200-400 nm. Samples 1 and 3 each exhibit a higher surface hardness value (approx. 14.2 GPa for each) while Sample 2 exhibits a low surface hardness value (approx. 11.1 GPa). The enhanced hardness value demonstrates the improved wear resistance of the material. Thus, this method allows improving wear resistance of the oxide ceramic surface layer formed by the zirconium alloy having a high hafnium content.

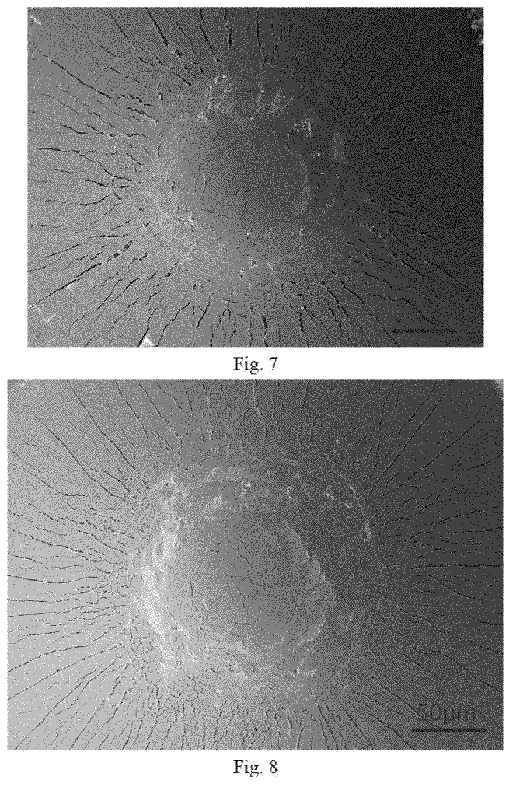

FIGS. 7 and 8 show indentations of Samples 2 and 3 created by a Rockwell diamond indenter under a load of 60 kg respectively, to characterize anti-crushing properties of the oxide ceramic surface layers. As can be seen, the indentation created on Sample 2 was deeper and broader than that on Sample 3, demonstrating the ability of the above method to improve the anti-crushing properties of a zirconium alloy with a high content of hafnium element.

### Embodiment 2

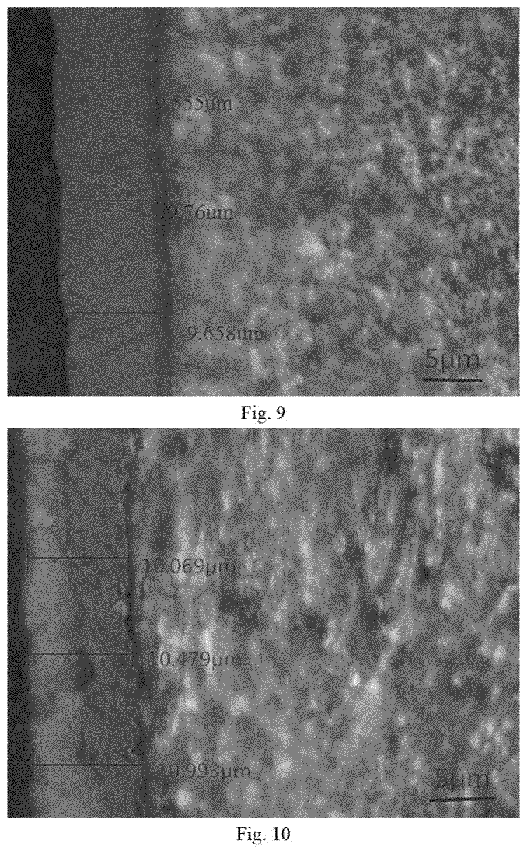

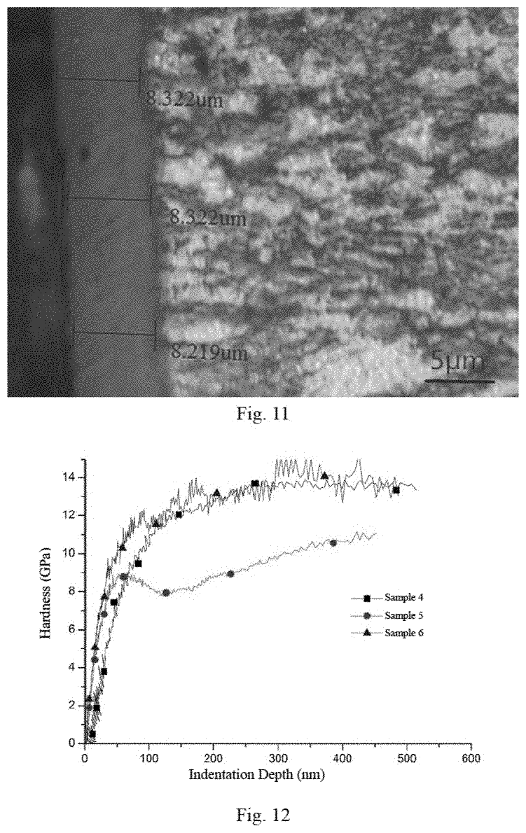

Samples 4 and 5 each with an oxide ceramic surface layer were prepared by heating zirconium alloys with hafnium contents of <0.005% and about 1.8% to 600° C. and maintaining them at the temperature for 4 h in air respectively. Cross-sectional morphologies of Samples 4 and 5 were observed with a metallurgical microscope, and corresponding metallurgical microscope images were shown respectively in FIGS. 9 and 10. In each of the images, there were an oxide ceramic layer on the left, an oxygen-rich diffusion layer in the middle and a metal substrate on the right. Compared with Sample 5, Sample 4 has an oxide ceramic surface layer possessing a high interior compactness, a tight bonding to the substrate without any crevice in the interface, as well as a thickness of about 9.66 μm. The Sample 5 has an oxide ceramic layer possessing a relatively large thickness of about 10.51 μm, a low interior compactness as well as numerous micro-cracks, even the tendency of cracking. In addition, the oxide ceramic layer of Sample 5 has a poor bonding to the substrate with distinct crevices. Both the oxide ceramic surface layer and the oxygen-rich diffusion layer of Sample 5 were removed by removing a surface thickness of about 12 μm of the sample through mechanical grinding, polishing or another means. Subsequently, Sample 5 was again heated at 600° C. in air for 4 h to obtain the Sample 6. The metallurgical cross-sectional morphology of Sample 6 was shown in FIG. 11. The morphology of oxide ceramic layer of Sample 6 is similar to that of oxide ceramic layer of Sample 4. Thus, after treated by the method of present application, the oxide ceramic layer for the formed Sample 6 has a thickness of about 8.29 μm, and obtains a considerably improved quality, thereby enabling to achieve the low hafnium content similar to that of Sample 4

FIG. 12 shows hardness curve graphs of the oxide ceramic surface layers of Samples 4 to 6. The hardness curve graph is measured by a nanoindentation instrument in a CSM (Continuous Stiffness Measurement) mode with an indentation depth of up to 500 nm and the plotted values were average hardnesses measured at indentation depths in the range of 200-400 nm. Samples 4 and 6 each exhibit a high surface hardness value (approx. 14.0 GPa and 13.6 GPa respectively) while Sample 5 exhibits a low surface hardness value (approx. 9.7 GPa). The higher hardness value means the better wear resistance of the material. Thus, this method is able to further improve wear resistance of the oxide ceramic surface layer formed by the zirconium alloy having a high hafnium content.

It is to be noted that, the medical implants as mentioned herein refer to implantable medical instruments that can be placed into surgically created or naturally occurring cavities in human bodies. Examples of the medical implants may include, but are not limited to, surgical implants such as artificial joints, (orthopedic, spinal, cardiovascular and neurosurgical) implants, structural prostheses, dentures and other artificial organs; implants made of metal materials (including stainless steel, cobalt-based alloys, titanium and alloys thereof and shape memory alloys), polymers, high molecular materials, inorganic non-metallic materials, ceramic materials, etc.; implantable instruments such as implantable orthopedic instruments, implantable aesthetic and plastic surgical instrument and materials; implantable appliances such as bones (plates, screws, pins, rods), intra-spinal fixation devices, staplers, patellar concentrators, bone wax, bone repair materials, plastic surgical materials, heart or tissue repair materials, intraocular filling materials, nerve patch, etc.; interventional instruments such as interventional catheters, stents, embolization and other devices; and orthopedic (orthopedic) surgical instruments such as scalpels, drills, scissors, forceps, saws, chisels, files, hooks, needle slickers, active instruments, extremity extension braces, multi-purpose unilateral external fixation devices and other instruments for orthopedic (orthopedic) surgical use.

Finally, it is to be noted that the above embodiments are provided merely to illustrate the technical solution of present application and are not intended to limit it in any way. Although the present application has been described in detail with reference to the above embodiments, modifications to those embodiments are still possible, or equivalent substituents of all or some of the technical features thereof can be made by those of ordinary skill in the art. Such modifications substituents do not cause the essence of corresponding technical solution to depart from the protection scope of the various embodiments of the present application.

1. A method of treating a zirconium alloy, comprising a step of performing a surface layer oxidation and removal treatment on the zirconium alloy, wherein the surface layer oxidation and removal treatment comprises: performing an oxidation treatment on a surface layer of the zirconium alloy to obtain an oxide surface layer; and performing a removal treatment to the oxide surface layer to expose a metal substrate.

2. The method of claim 1, wherein the zirconium alloy has an initial content of hafnium element ranging from 0.5 wt % to 8 wt %.

3. The method of claim 1, wherein the oxidation treatment is conducted at a temperature of 500° C. to 700° C. and a treatment time of 0.5 h to 10 h.

4. The method of claim 1, wherein the oxide surface layer is removed by means of grinding, fine machining, mechanical polishing, vibratory polishing or any combination thereof.

5. The method of claim 1, wherein a thickness of the oxide surface layer removed in the removal treatment ranges from 1 μm to 20 μm.

6. The method of claim 5, wherein the thickness of the oxide surface layer removed in the removal treatment ranges from 3 μm to 12 μm.

7. The method of claim 1, further comprising repeating the step of performing a surface layer oxidation and removal treatment for 1 to 5 times.

8. A method for producing an oxide ceramic layer on a surface of a zirconium alloy, comprising treating the zirconium alloy with the method of claim 1; and performing an oxidation treatment on a surface of the exposed metal substrate.

9. The method of claim 8, wherein the oxide ceramic layer has a content of hafnium element ranging from 0.3 wt % to 6 wt %.

10. A material for use in medical implants, prepared by the method of claim 1 and comprising a metal substrate, an oxygen-rich diffusion layer and an oxide ceramic layer, the metal substrate made of a zirconium alloy, wherein a content of hafnium element in the metal substrate is higher than a content of hafnium element in the oxide ceramic layer.

11. The material of claim 10, wherein the content of hafnium element in the metal substrate ranges from 0.5 wt % to 8 wt %, and the content of hafnium element in the oxide ceramic layer ranges from 0.3 wt % to 6 wt %.
